(19) United States
(12) Patent Application Publication
(10) Pub. No.: US 2025/0066414 A1
(43) Pub. Date: Feb. 27, 2025

(54) PROCESSES AND SYSTEMS FOR FLOW-THROUGH OLIGONUCLEOTIDE SYNTHESIS

(71) Applicant: PEPTISYSTEMS AB, Uppsala (SE)

(72) Inventors: Lars-Åke HOLMBERG, Uppsala (SE); Ulf TEDEBARK, Järfälla (SE); Rannar SILLARD, Enebyberg (SE)

(73) Assignee: PEPTISYSTEMS AB, Uppsala (SE)

(21) Appl. No.: 18/692,033

(22) PCT Filed: Sep. 15, 2022
(86) PCT No.: PCT/SE2022/050812

§ 371 Date: Mar. 14, 2024

(30) Foreign Application Priority Data
Sep. 16, 2021 (SE) 2151135-7

(51) Int. Cl. C07H 21/00; B01J 19/00

(52) U.S. Cl. CPC C07H 21/00; B01J 19/0046; B01J 2219/00722

(57) ABSTRACT

This invention relates to processes and systems for flow-through solid-phase oligonucleotide synthesis. The process comprises the steps of detritylation. coupling. oxidation, and capping. The process comprises at least one step of reconsolidating the synthetic resin that occurs after the detritylation step. The step of reconsolidating the synthetic resin comprises a first fluidizing step followed by a repacking step. In the fluidizing step solvent flow from the bottom of the column to the top, and in the repacking step solvent flow from the top of the column to the bottom.

### FIELD OF INVENTION

This invention relates to processes, methods and systems for solid phase oligonucleotide synthesis.

### BACKGROUND

Oligonucleotides in nature are DNA or RNA molecules. They have generated great interest due to their use in drug discovery, therapeutic modalities, molecular diagnostics, etc. To date there are more than 10 FDA approved oligonucleotide drugs and most of them have been approved in 2016 or later. Oligonucleotides can be synthesized in solution or in a flow-through process wherein a synthetic resin is provided in a column. Protected nucleosides or nucleotides are attached to the resin via a linker. In a first step of the reaction the protected nucleoside or nucleotide are deprotected by a deprotecting agent. A second nucleotide that is dissolved in a solvent is added to the column so that the second nucleotide is coupled to the first nucleotide or nucleoside. These steps can be repeated to produce an oligonucleotide chain or sequence. After the desired oligonucleotide sequence is synthesized, the oligonucleotide is separated from the resin.

The synthetic resin may shrink and swell during the process depending on the type of resin, the ratio between the resin amount versus the amount of the growing oligonucleotide and the solvents used in the process. Swelling and shrinkage of the resin may lead to increased phase heterogeneities of the same during oligonucleotide synthesis. Phase heterogeneity can lead to a non-optimal flow through the resin, i.e. e.g., that the front is not horizontal, or perpendicular, against the column wall, and therefore the flow is not homogenous through the entire resin. This in turn will have a negative impact on the synthesis and the quality of the synthesized oligomer.

U.S. Pat. No. 5,641,459 discloses a machine for synthesizing oligonucleotides that has individual modulus for connection to each of a number of different monomers and to other fluids used in the synthesizing process.

U.S. Pat. No. 6,469,157 B1 discloses an apparatus for preparing polynucleotides on a solid support in a reactor comprising a column containing an immobilized solid support that is functionalized for polynucleotides synthesis.

EP 3650455A1 discloses a novel solvent that can be used as an alternative to toluene in the step of deprotection in the method of solid-phase nucleic acid synthesis, and a method in which removing of the protecting group from the protected nucleoside phosphoramidite is carried out in a solution comprising an acid with a pKa of 0.2 to 0.8 and acetonitrile.

In the prior art there is a need for improved processes and systems for oligonucleotide synthesis that overcomes problems of the prior art. There is also a need for processes and systems for oligonucleotide synthesis that prevent or minimize the negative effects of resin phase heterogeneities.

### SUMMARY

The object of the present invention is to provide a process and a system for oligonucleotide synthesis that overcomes the drawbacks of the prior art systems and processes. This is achieved by the process as defined in claim 1, the system as defined in claim 16, and the method as defined in claim 22.

According to one aspect of the invention there is a flow-through process for solid-phase oligonucleotide synthesis in which a first hydroxyl-protected sequence unit attached to a synthetic resin via chemical linkage is provided in a column. The process comprises steps (i)-(iii), repeated at least once as a cycle, of
i. by detritylation, unprotecting the hydroxyl group of the first sequence unit attached to a synthetic resin;
ii. providing a liquid reaction solution comprising at least one second sequence unit; and
iii. passing the reaction solution over the synthetic resin with the at least one first sequence unit attached, whereby the at least one second sequence unit is coupled to the first sequence unit attached to the resin, and the reaction solution consolidates the resin to a more dense state in the column than provided in step (i);
   within which process the step of removing a protecting group from the first sequence unit attached to a synthetic resin is performed in an acidic solution, and wherein the process comprises at least one step of reconsolidating the resin.

In one aspect of the invention there is a system for flow-through solid-phase oligonucleotide synthesis. The system comprises at least one column arranged to be packed with synthetic resin; at least a first pump; at least a first reservoir for monomers and reagents; at least one detector, and at least one processor. Tubing connects the first reservoir to the column, and the first pump and valves are arranged to direct the flow in the system. The processor is in communicative connection at least with the first pump, the valves, and the detector. The system is arranged to provide a fluidizing flow path arranged to direct a flow of solvent from the first reservoir to the bottom of the column so that solvent pass through the column from the bottom of the column to the top. The system is arranged to provide repacking flow path arranged to direct a flow of solvent from the first reservoir to the top of the column so that solvent pass through the column from the top to the bottom. The system is arranged to in one synthesis cycle first flow solvent through the fluidizing flow path followed by flowing solvent through the repacking flow path.

In one aspect of the invention there is a method for performing flow-through solid-phase oligonucleotide synthesis using a system for flow-through solid-phase oligonucleotide synthesis. The system comprises at least one column arranged to be packed with synthetic resin; at least a first pump; at least a first reservoir for monomers and reagents; at least one detector, and at least one processor. Tubing connects the first reservoir to the column. The first pump and valves are arranged to direct the flow in the system. The processor is in communicative connection at least with the first pump, the valves, and the detector. A synthetic resin is arranged in the column, and a first sequence unit is attached to the synthetic resin via chemical linkage. The method comprises at least one step of reconsolidation, during which the system is arranged to first fluidize the synthetic resin arranged in the column by flowing a solvent from the bottom of the column to the top via the fluidizing flow path in a fluidizing step. The fluidizing step is followed by a repacking step in which the system is arranged to repack the synthetic resin arranged in the column by flowing a solvent from the top of the column to the bottom using the repacking flow path.

### DESCRIPTION OF DRAWINGS

FIGS. 1 a) and b) are flow-charts of processes according to the invention;

FIGS. 2 a) and b) are schematic illustrations of systems according to the invention;

FIG. 3 is a schematic illustration of a system according to the invention;

FIG. 4 is a schematic illustration of a system according to the invention;

FIG. 5 is a block-scheme according to the invention; and

FIGS. 6 a) is an HPLC chromatogram from an example according to the invention, and b) is an HPLC chromatogram from a comparative example.

### DEFINITIONS

The terms used herein have their normal meaning in the art unless otherwise specified.

The term “flow-through” is used to describe a process wherein a solution comprising reagents, and/or solvents, and/or additives, etc. is passed over/through a column, the term includes both single pass (i.e. wherein the solution(s) are passed one time over the column) and re-circulation (i.e. wherein the solution(s) are re-circulated through the system and passed over the column again at least a second time).

The term “sequence unit” refers to a compound used as a unit in a sequence. A sequence unit may store/carry information. Examples of sequence units includes nucleoside(s), nucleotides, phosphorodiamidate morpholino oligomer (PMO), bicyclic/tricyclic nucleoside and nucleotide analogues such as LNA, cEt, ENA, GNA, TNA, FNA etc. Examples of sequences include DNA, RNA, and analogues of those e.g., 2′ modification such as MOE, OMe, F etc. Sequence units may also be any non-nucleotide, non-nucleoside based compound in a sequence together with DNA, RNA or analogues of those. A first sequence unit may also be part of the universal support.

The term “nucleotide” refers to a nucleic acid subunit, a nucleotide comprises a sugar group, a base, and a phosphate group or any other phosphate analogue where phosphorothioate being one of the most common.

The term “nucleoside” refers to a compound comprising a sugar and a base.

The term “base” refers to nitrogenous bases. There are a number of different bases available that can be used, the five most common for DNA and RNA are adenine, cytosine, guanine, and thymine/uracil (abbreviated as A, C, G, and T/U). Other examples are methyl-uracil (MeU) and methyl-cytosine (MeC), or any other functional group(s) attached to or replaced/included in the base structure.

The term “nucleic acid” includes both DNA and RNA, and any analogue to DNA and RNA. The analogues differ from DNA and RNA in the structure of the backbone.

The term “synthetic resin” includes organic compounds or solid-phases capable of forming a packed bed in a column, a synthetic resin comprises for example a polymer material based on for example polystyrene or divinylbenzene but also other solid-phases or combinations of solid-phases having any degree of cross-linking or pore structure.

The term “reagent” refers to any compound, sequence unit, reagent, solvent that are used in the generation of a growing nucleotide sequence in the flow-through system.

The term “about” as used herein is to be understood to encompass variations of ±10%, or ±5%, or ±1%.

The term “fluidizing” refers to when more than about 1% of the bottom surface of the synthetic resin is no longer in contact with the bottom of the column, this stage can be obtained for example by applying a flow through the column from the bottom and up.

Throughout the specification the terms “oligonucleotide”, “polynucleotide”, and “oligomer” are used interchangeably and refer to a chain or sequence of sequence units linear or branched, i.e. a sequence e.g., nucleic acids such as DNA/RNA or analogues thereof.

Throughout the specification, unless stated otherwise, “sequence units” such as “nucleoside(s)”, “nucleotide(s)”, “oligonucleotide(s)”, and “polynucleotide(s)” refers to those having activating, and/or protecting groups as suitable.

The term “detritylating” or “detritylation” refers to removal of protecting group(s) to allow adding of an additional sequence unit. Detritylation includes removal of dimethoxytrityl or monomethoxytrityl or any other protecting group to allow addition and coupling of an additional sequence unit.

The term “linearly scalable” have the meaning of a synthesis process wherein the synthesis conditions in a smaller or larger scale can be transferred to an alternative size of scale giving a similar or identical synthesis outcome.

The term “conditional threshold” is used in the meaning of a predefined value of at least one parameter set in a control software to allow the previous step(s) to be ended and a subsequent step to start.

‘ACN’ is an abbreviation for acetonitrile and used in its conventional meaning in the art.

‘DCA’ and ‘TCA’ are abbreviations for dichloroacetic acid and trichloroacetic acid respectively and used in its conventional meaning in the art.

‘PAT’ is an abbreviation for Process Analytical Technology and used in its conventional meaning in the art.

‘CPP’ is an abbreviation for Critical Process Parameters and used in its conventional meaning in the art.

‘CQA’ is an abbreviation for Critical Quality Attributes and used in its conventional meaning in the art.

‘NIR’ is an abbreviation for Near Infrared and used in its conventional meaning in the art.

‘UV/Vis’ is an abbreviation for Ultraviolet/Visible and used in its conventional meaning in the art.

### DETAILED DESCRIPTION

Terms such as “top”, “bottom”, “upper”, “lower”, etc are used merely with reference to the geometry of the embodiment of the invention shown in the drawings and are not intended to limit the invention in any manner.

Methods and processes for synthesizing oligonucleotides includes phosphoramidite, phosphotriester, and H-phosphonate methods and other similarly methods, all of which are generally known within the field. The systems and processes described herein are described in connection with a phosphoramidite method, it should however be understood that the processes and the systems can be utilized using other synthesis methods as well.

As discussed in the background oligonucleotides, or oligomers, can be synthesized in a flow-through process in which the growing oligonucleotide is immobilized on a resin arranged in a column. A flow-through system comprises column(s), valve(s) and pump(s) all in fluid communication with each other using tubes. The pump(s) are arranged to pump liquid through the system and over the column. Prior to the start of the synthesis the column is filled with a resin to which a first nucleotide or nucleoside is attached either directly via covalent linkage or via a linker unit, also referred to as a universal support. The linker unit may be a sequence unit that remain at the resin after cleavage and is hence not part of the final sequence unit after cleavage.

In a first aspect of the invention there is a flow-through process for solid-phase oligonucleotide synthesis in which a first hydroxyl-protected sequence unit is attached to a synthetic resin via chemical linkage is provided in a column. The process comprises steps (i)-(iii), repeated at least once as a cycle, of:
i. by detritylation, unprotecting the hydroxyl group of the first sequence unit attached to a synthetic resin;
ii. providing a liquid reaction solution comprising at least one second sequence unit; and
iii. passing the reaction solution over the synthetic resin with the at least one first sequence unit attached, whereby the at least one second sequence unit is coupled to the first sequence unit attached to the resin, and the reaction solution consolidates the resin to a more dense state in the column than provided in step (i).

The second sequence unit is coupled to the first sequence unit attached to the synthetic resin. The step of removing a protecting group from the first sequence unit attached to a synthetic resin, i.e. the detritylation step, is performed in an acidic solution. The process comprises at least one step of reconsolidating the synthetic resin.

As the skilled person will appreciate, in case of a cyclic process, the term “a first sequence unit” will refer to the latest added sequence unit.

The term “reaction solution” encompasses any reagent, reagents, sequence unit(s), solvents, additives etc. required for the growing sequence.

The step of reconsolidating the synthetic resin comprises fluidizing the synthetic resin by passing a solvent from the bottom of the column and up, or in the countercurrent direction, and a step of repacking the synthetic resin. The step of repacking the resin follows after the step of fluidizing the synthetic resin. In the step of repacking the synthetic resin, a solvent is passed from the top of the column and down, or in the current direction.

In other words, in a first aspect of the invention there is a flow-through process for solid-phase oligonucleotide synthesis in which a first hydroxy-protected sequence unit is attached to a resin via chemical linkage. The process comprises steps (i)-(iii), repeated at least once as a cycle, of
i. by detritylation, unprotecting the hydroxyl group of the first sequence unit attached to a synthetic resin;
ii. providing a liquid reaction solution comprising at least one second sequence unit; and
iii. passing the reaction solution over the synthetic resin with the at least one first sequence unit attached, whereby the at least one second sequence unit is coupled to the first sequence unit attached to the resin, and the reaction solution consolidates the resin to a more dense state in the column than provided in step (i).

The second sequence unit is coupled to the first sequence unit attached to the synthetic resin. The step of removing a protecting group from the first sequence unit attached to a synthetic resin, i.e. the detritylation step, is performed in an acidic solution. The process comprises at least one step of fluidizing the resin by passing a solvent from the bottom of the column and up, or in the countercurrent direction, and a subsequent step of repacking the resin. In the step of repacking the resin a solvent is passed from the top of the column and down, or in the current direction.

In one embodiment of the invention the step of reconsolidation comprises fluidizing the resin.

In one embodiment of the invention the sequence units are nucleotide(s) or nucleoside(s).

A process according to the first aspect may be used to synthesize an oligonucleotide, an oligomer, a DNA molecule, an RNA molecule, analogues of DNA/RNA, or any other macromolecule that comprises a sequence or chain of sequence units e.g., nucleic acids. All embodiments, aspects, and variants described herein may be in connection with all types of macromolecules described above.

Prior to starting an oligonucleotide synthesis, a synthetic resin is provided in a column. As discussed above, a first sequence unit, or nucleic acid, or monomer is attached to the synthetic resin, either directly or via a linker unit. The synthetic resin has a particle like appearance with different degrees of developed pore structures, and is typically a polymer or a polymeric material with a certain degree of crosslinking, such as 70% or less, or 50% or less, or 30% or less or 10% or less. The synthetic resin may be any type of synthetic resin that is used in the field, for example, polystyrene (PS), divinylbenzene (DVB), or a mixture such as PS-DVB etc. The synthetic resin may have any shape or form, such as plate, particulate, fibrous, etc. In one embodiment the resin is cross-linked polystyrene, e.g. NittoPhase or Primer Support 5G or similar. A synthetic resin may swell and/or shrink in contact with various solvents such as in an oligonucleotide synthesis or process. Swelling and shrinkage of the synthetic resin arranged in a column may lead to the formation of a non-homogenous resin areas in the column, or the formation of heterogeneities in the resin. Heterogeneities in the resin may also arise in other ways. Also other types of resins, for example a mixture of a polymer and another material, may experience heterogeneities in an oligonucleotide synthesis process.

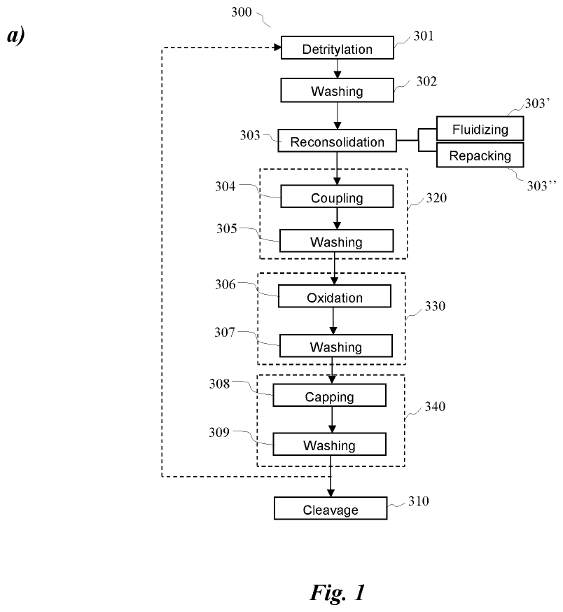

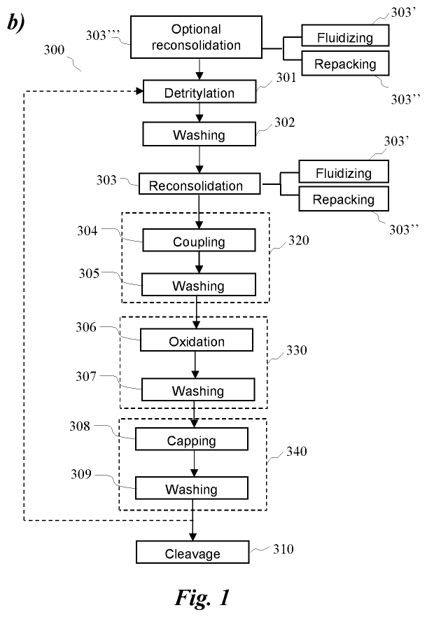

A flow-chart for a general oligonucleotide process or synthesis according to the invention is shown in FIG. 1a. In a first step a first sequence unit is deprotected through detritylation 301. In the detritylation step 301 a 5′-dimethoxytrityl protecting group is removed from a first sequence unit. As a result, a hydroxyl group becomes available for coupling. In the case that the first sequence unit is more than one sequence unit (i.e. that a chain or sequence is attached to the resin), the hydroxyl group of the e.g., last sequence unit in the sequence becomes available for coupling. In some cases, a functional group such as a hydroxyl, amine etc. ready for further functionalization may be available in any other position than the last sequence unit to allow for e.g., branched oligonucleotides, oligonucleotide derivatives, other sequences, other sequence units, other macromolecules.

Following the detritylation there is a first washing step 302 during which a solvent is passed through the column. Required wash volumes are dependent on physical parameters such as column length and diameter, bed height, diffusion, etc. and can be determined by persons skilled in the art. Required wash volumes can also be determined via fulfilment of conditional thresholds as determined by the skilled person.

After the detritylation step 301 the synthetic resin in the column may shrink and/or become non-homogenous and/or comprise heterogeneities, as discussed above. Therefore, following the first washing step 302 there is a first reconsolidation step 303. The first reconsolidation 303 step comprises two steps: a first step of fluidizing 303′ the resin followed by a step of repacking 303″ the resin. In the fluidizing step 303′ solvent e.g., ACN is first passed through the column in a counter-current, or opposite flow, direction, i.e. from the bottom of the column and up. Solvent is passed in counter-current direction through the column until the synthetic resin, or resin bed, is fluidized. Hence, until a fluidized bed is formed in the column. Once a fluidized bed is formed, a repacking step 303″ follows. In the repacking step 303″ solvent is passed through the column in the opposite direction, i.e. in the current direction or from the top of the column to the bottom.

An oligonucleotide process or synthesis may additionally comprise an optional reconsolidation step 303′″ as illustrated in FIG. 1b. Such an optional reconsolidation step 303′″ is performed prior to the detritylation step 301. An optional reconsolidation step 303′″ comprises a fluidizing step 303′ followed by a repacking step 303″, i.e. as the reconsolidation step 303 described above.

In one embodiment the first step of reconsolidating 303 the synthetic resin is performed after the detritylation 301. As described above the first reconsolidation step 303 comprises a first step of fluidizing the resin 303′ wherein the solvent is passed from the bottom of the column to the top, followed by a step of repacking 303″ the resin during which the solvent is passed from the top of the column to the bottom. A second reconsolidation step may be performed after the fourth washing step 309 discussed below. Additional reconsolidation steps may be performed after any of the washing steps 302;305;307;309 in the synthesis (all washing steps are discussed further down).

Following the first reconsolidation step 303 there is a reaction step 320. The reaction step 320 comprises a coupling step 304 followed by a second washing step 305. In the coupling step 304 at least one second sequence unit is passed over the resin (through the column). During the second washing step 305 a solvent, e.g. acetonitrile (ACN), propionitrile, dimethylformamide (DMF) followed by ACN or any other solvent or solvent combination(s) suitable to facilitate removal of reagents, by-products, excess sequence units, additives etc. is passed over the column. The coupling step 304 may be repeated one or several times, so that the at least one second nucleotide or nucleoside may be re-circulated over the resin. Prior to the coupling 304 the at least one second nucleotide or nucleoside may be activated by an activating agent, e.g., BTT, ETT, activator 42. In the coupling step 304 phosphorous in the second sequence unit forms a covalent bond with a hydroxyl group in the resin-bound first sequence unit. It is an advantage to have anhydrous reaction conditions during the coupling step 304. Anhydrous conditions may result in a higher yield and/or higher purity of the formed oligonucleotide or oligomer.

An oxidation/thiolation 330 follows the reaction step 320. The oxidation/thiolation step 330 comprises an oxidation step 306 and a third washing step In 307. the oxidation step 306 an oxidating/thiolating/sulfurization agent is added to the column 304. The oxidating/thiolating/sulfurization agent convert the newly formed trivalent phosphorous to a pentavalent (e.g., phosphorothioate, phosphodiester). Secondly the remaining oxidating/thiolating/sulfurization agent is flushed in a third washing step 307. In the third washing step 307 solvent is passed through the column.

After the oxidating/thiolating/sulfurization agent is flushed away a full capping step 340 follows. The full capping step 340 comprises a capping step 308 and a fourth washing step 309. In the capping step 308 a capping agent is added to the column. The capping agent block unreacted hydroxyl groups and prevent them from reacting in the following steps. The introduction of the capping agent is followed by a fourth washing step 309. In the fourth washing step 309 solvent is passed through the column. The capping step could be optional. The capping step may also be included in the oxidation/thiolation step.

The steps of detritylation 301, reconsolidation 303, reaction 320, oxidation/thiolation 330, and full capping 340 are repeated until an oligomer or oligonucleotide or polynucleotide comprising a desired nucleic acid sequence is synthesized. After the desired sequence is synthesized the newly formed sequence (e.g. the newly formed oligo-or polynucleotide) is removed from the resin in a cleavage step 310. The desired sequence may be eliminated from the resin by any conventional technique available. The oligonucleotide or polynucleotide may comprise up to 25, or up to 40-50 or up 70-80, or up to 100 or more sequence units, or nucleotides or nucleosides.

An exemplary oligonucleotide synthesis cycle involves the repetition of the steps described above, i.e. detritylation 301, first washing step 302, a first reconsolidation 303, reaction 320, oxidation 330, and capping 340. After the desired sequence has been synthesized it is eliminated from the resin in a cleavage step 310. Either prior to, during or directly after the elimination from the resin, any protecting group or protecting groups may also be cleaved from the oligomer, or oligonucleotide.

It is possible to monitor the first reconsolidation step by using a glass (or any other suitable transparent material) column and visually monitor the resin arranged in the column. After a successful first reconsolidation step 303 the resin will appear more homogenous or homogenous in the column. In one embodiment the first reconsolidation step 303 is performed so that the flow rate during the fluidizing step is 25-75 cm/h, and the fluidizing step is continued for 0.25-0.75, or 0.25-1 column volumes, and the flow rate during the repacking step is 100-200 cm/h, and the repacking is continued for 0.25-0.75, or 0.25-1 or more column volumes.

In a process as described herein comprising more than one synthesis cycle and a second reconsolidation step performed after the full capping 340 (or after the fourth washing step 309) it is possible to monitor the second reconsolidation step by following the red front formed in the upcoming/following detritylation step 301. In the case of a proper second reconsolidation step the red front or line formed during the detritylation step 301 will appear as a generally straight line perpendicular in relation to the column wall. This in contrast to when a second reconsolidation step 303 has not been carried out in which case the line may be non-linear, or non-perpendicular, or the front will not form a line.

As described above the nucleoside(s) or nucleotide(s) are activated prior to the reaction step 320. In one embodiment the sequence unit(s) (e.g. the nucleoside(s) or nucleotide(s)) are activated in a separately defined mixing chamber. In one embodiment the sequence unit(s) (e.g. the nucleoside(s) or nucleotide(s)) are activated in the tubing of the system. Activation of the nucleoside(s) or nucleotide(s) inside the tubing of the system may be referred to as in-line activation. The in-line activation may occur before reaching the column or inside the column.

Any suitable solvent or solvents may be used in a process described herein. Commonly, acetonitrile (ACN) is used as a solvent in the washing steps 302;305;307;309, and an acidic solution is used in the detritylation step 301 wherein the protecting group is removed from the first nucleoside(s) or nucleotide(s) attached to the resin. In one embodiment the pKa of the acidic solution used in the detritylation step is at a value so that it can release the protecting group e.g., DCA or TCA for dimethoxytrityl, with pKa values of e.g., 1.35 for DCA, 0.66 for TCA. In one embodiment the pKa of the acidic solution used in the detritylation step is 0.2-3, or 0.2-2, or 0.2-1.5, or 0.2-0.8.

In one embodiment the reaction solution is passed over the resin at a flow rate and in a volume that allow for the second sequence unit in the reaction solution to be in contact with at least 99%, or 98%, or 97%, or 96%, or 95%, or 90%, of the immobilized sequence units.

In a flow-through process for solid-phase synthesis of oligomers or oligonucleotides as described herein a solution comprising a second sequence unit are passed over a column comprising at least one immobilized first sequence unit. The solution may be passed over the column once or re-circulated and passed over the column more than once, such as twice, or three times, or more. In the case of re-circulation different reagents and/or additives may be added to the solution that are being re-circulated. A skilled person can determine the appropriate number of cycles depending on the type of oligonucleotide that is being synthesized, the desired outcome, the type of reagents, etc.

In one embodiment heating is applied to the at least one second sequence unit prior to being passed over the synthetic resin. The at least one second sequence unit may be heated at least 1° C. prior to being passed over the synthetic resin.

In the present context heating may refer to a heating of 1° C., or 3° C., or 5° C. or more. A person skilled in the art can determine the heating level depending on the reaction, type(s) of sequence unit(s), or nucleic acid(s)/nucleotide(s)/nucleoside(s), type(s) of reagents etc. It is an advantage with heating the sequence unit(s) or nucleoside(s) or nucleotide(s) and possible other reagents such as the capping agent(s), oxidation agent(s), etc. in that it may increase the reaction rate. Heating can be used in the process of oligonucleotide synthesis in order to increase the reaction rate of one or several reaction steps.

The different steps of the process described herein may be monitored using one, two, or several detectors. In one embodiment the progress of at least one step of the process is monitored by at least two detectors. In one embodiment the progress of the process is continuously monitored. In one embodiment the progress of the process is monitored by spectroscopy, preferably near-infrared (NIR) spectroscopy.

As described above one, a few, or all parts/steps of the process described herein may be monitored either continuously or in a non-continuous way. Hence, the detritylation 301, the first reconsolidation 303, the reaction step 320, the oxidation/thiolation step 330, the full capping step 340 and/or the different washing steps 302;305;307;309 may be monitored. The detector may be a NIR and/or UV/Vis detector. Continuous monitoring can provide information about the progress of the different steps: detritylation 301, reconsolidation 303, coupling 304, oxidation 306, capping 308, the different washing steps 302;305;307;309, etc. Such information may for example be when the present, on-going step, is finished and the next step may begin, i.e. reaching a conditional threshold. The information from the monitoring can also be used to change the synthesis protocol depending on the output from the continuous monitoring. The process can be monitored in different ways such as for example in-line, or on-line, or both. The process may be monitored in ways that allows for continuous monitoring.

As described above, activation of the sequence unit(s) or nucleoside(s) or nucleotide(s) may be performed in a separately defined mixing chamber. In such case the mixing chamber may be emptied and cleaned between the cycles using dilution with a solvent and the progress of the cleaning may be monitored using a detector, such as a UV/Vis or an NIR detector, or any other type of detector that is suitable as determined by the skilled person. The emptying and cleaning of the mixing chamber may be performed while the resin in the column is being cleaned either running the two processes independently and simultaneously or by passing the cleaning solvent first through one of these units and then through the other (i.e. first through the mixing chamber and then over the column or vice versa).

Washing of the mixing chamber can be combined with washing the column by passing a washing solvent e.g. ACN sequentially first to the mixing chamber and thereafter over the column.

In another embodiment, the outflow of the mixing chamber can be monitored using a gas bubble detector.

The different washing steps 302;305;307;309 may be performed using displacement technique. Washing of the column may be monitored in-line and in real-time and continued until a conditional threshold has been reached. Such threshold is determined by a person skilled in the art. The monitor may be a UV/Vis or NIR monitor/detector or any other suitable kind of detector.

In one embodiment the process uses software controlled real-time conditional monitoring and enables the use of Process Analytical Technology (PAT) to measure Critical Process Parameters (CPP) which affect Critical Quality Attributes (CQA).

Process Analytical Technology (PAT) is an important tool for quality insurance in the biotechnology and pharmaceutical production industries. It is an advantage that a process enables PAT since PAT is important to meet the requirements of different regulatory bodies such as the US Food and Drug Administration (FDA) for example. The use of UV/Vis and/or NIR spectroscopy to monitor the different steps of the reaction may enable the use of PAT.

The use of PAT may allow the user to choose which of the parameters e.g. time, absorbance of light at certain wavelengths, temperature, presence or absence or both of certain intermediates, by-products, etc. that control the process. Defined parameters may be documented and logged in the control software according to 21 CFR part 11 compliance.

In one embodiment the process is linearly scalable. The process described herein may be linearly scalable, i.e. the liquid flow rate in the column (in cm/h) may be the same independent of the size/proportions of the column, and hence the same linear flow rate may be used in all scales, e.g. μmol, mmol, mol etc. This means that a process described herein may be scaled up (or scaled down) using the same relative excess of sequence unit(s) or nucleoside(s) or nucleotide(s) in a smaller scale (e.g. μmol) as in a larger scale (e.g. mmol, mol), or vice versa (i.e. transfer of conditions from a larger to a smaller scale), due to the use of the same linear liquid flow rate in the various steps. Therefore, the process can be optimized in small scale prior to being upscaled or downscaled, which may save both time, reagents, solvents, and reduce waste. The process may give similar or identical results, e.g. yield and purity, in the scaled version as in the scale where the parameters were optimized. As understood by persons skilled in the art for a process to be linearly scalable not all process parameters (e.g. pressure, temperature, etc.) and not all synthesis components (e.g. additives, solvents, etc.) must have a linear relationship between different synthetic scales.

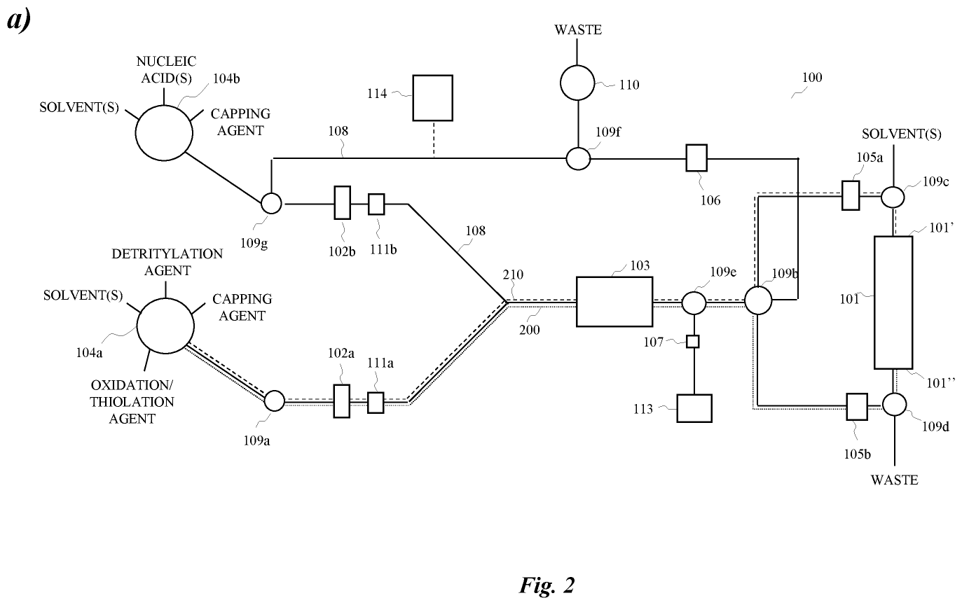

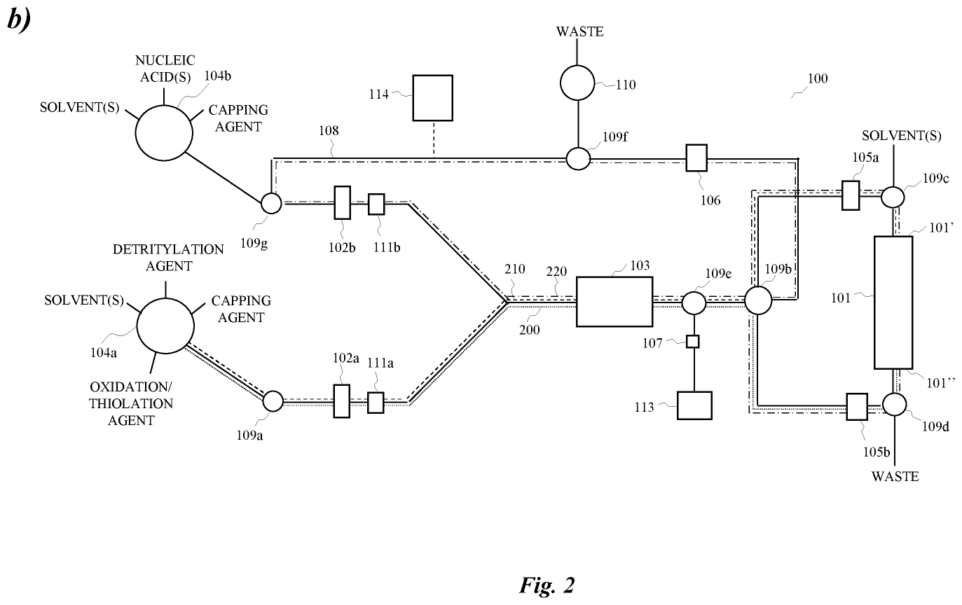

In a second aspect of the invention there is a system 100 for flow-through solid phase oligonucleotide synthesis. The system 100 comprises at least one column 101 arranged to be packed with synthetic resin, at least a first pump 102a, at least a first reservoir for solvents and reagents 104a, at least one detector 106, and at least one processor 114. Tubing 108 connects the first reservoir 104a to the column 101. The first pump 102a and valves 109a-d are arranged to direct the flow in the system 100. The processor 114 is in communicative connection at least with the first pump 102a, the valves 109a-d, and the detector 106. The system 100 is arranged to provide a fluidizing flow path 200 arranged to direct a flow of solvent from the first reservoir 104a to the bottom of the column 101′ so that solvent pass through the column 101 from the bottom 101′ of the column to the top 101″, and arranged to provide repacking flow path 210 arranged to direct a flow of solvent from the first reservoir 104a to the top of the column 101″ so that solvent passes through the column 101 from the top 101″ to the bottom 101′. The system 100 is arranged in one synthesis cycle first to flow solvent through the fluidizing flow path 200 followed by flowing solvent through the repacking flow path 210. Such a system is schematically illustrated in FIG. 2a.

In one embodiment when solvent passes through the bottom of the column 101′ to the top 101″ the third valve 109c is arranged provide a waste outlet, i.e. to let waste out of the system 100. Similarly, when solvent passes through the top of the column 101″ to the bottom 101′ the fourth valve 109d is arranged provide a waste outlet, i.e. to let waste out of the system 100.

In one embodiment the second valve 109b is arranged downstream the first reservoir 104a and upstream the column 101, as illustrated in FIG. 2a. The second valve 109b may be arranged to direct the flow to enter either the column 101 from the top 101′ or from the bottom 101″. In such way the second valve 109b may facilitate the fluidizing flow path 200 and the repacking flow path 210 by directing the flow.

In one embodiment the first valve 109a is arranged downstream the first reservoir 104a and upstream the first pump 102a, as illustrated in FIG. 2a. In such a configuration the first valve 109a may be arranged to select the type of liquid that should enter the system 100 and the first pump 102a from the first reservoir 104a, in particular if the first reservoir 104a is realized by a plurality of reservoirs. In other embodiment the function of selecting the type of liquid is realized by the first reservoir 104a so that the first pump 109a is realized by or integrated in the first reservoir 104a.

In one embodiment the system 100 is arranged to provide a recirculation flow path 220. The re-circulation flow path 220 is arranged to re-circulate solvent(s), reagents, etc. through the system 100, over the column 101. Such a system 100 is schematically illustrated in FIG. 2b. In other embodiments the re-circulation flow path 220 may be realized by the tubing 108 and/or the fluidizing flow path 200 together with the repacking flow path 210. The second pump 102b may be arranged so that it is in fluid communication with the re-circulation flow path 220, as illustrated in FIG. 2b. In such a configuration the second pump 102b may be arranged to regulate the flow in the re-circulation flow path 220.

The components used in a system 100 according to the invention are standard components used in the field and hence familiar to the skilled person.

The column 101 may be any type of column, such as a glass column, a column fabricated from stainless steel, etc. The column 101 is arranged substantially straight in the gravitational direction in the system 100 so that the column top 101″ is arranged facing substantially upward (towards the sky) and the column bottom 101′ is arranged facing substantially downward (towards the earth). In such way liquid flow from the top of the column 101″ to the bottom 101′ flow in the gravitational direction. The column 101 is arranged to be packed with synthetic resin.

The system 100 may comprise more than one reservoir 104a, such as two reservoirs (a first 104a and a second 104b) as illustrated in FIGS. 2-4. In such case the first reservoir 104a may comprise solvent(s), detritylation agent(s), and capping agent(s), and the second reservoir 104b may comprise solvent(s), sequence units or nucleic acid(s), and capping agent(s) or vice versa or in any other combination.

The system 100 may comprise more than one pump 102a such as two pumps: a first 102a and a second 102b, as illustrated in FIGS. 2-4, or more. Each pump 102a; 102b may be in fluid communication with at least one of the reservoir(s) 104 in a system 100, as illustrated in FIGS. 2-4 wherein a first pump 102a is arranged downstream and in fluid communication with the first reservoir 104a and a second pump 102b is arranged downstream and in fluid communication with the second reservoir 104b. The pump(s) 102 may be any device that can be used to drive a flow of liquid in a system 100, for example hydraulic pump(s), pneumatic pump(s), etc.

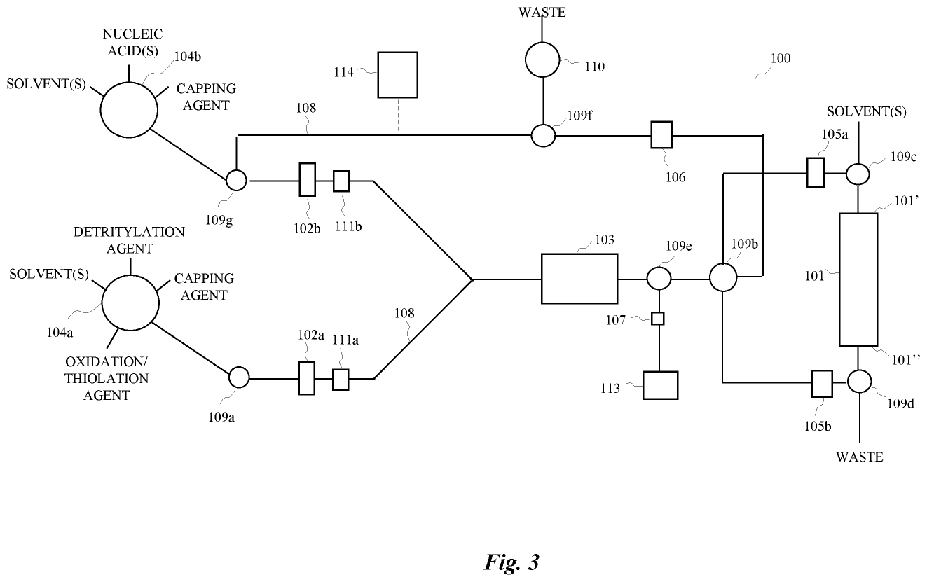

In one embodiment the fluidizing flow path 200 and the repacking flow path 210 is realized by the tubing 108, hence no extra tubing is necessary. Instead, the system 100 is arranged to first flow liquid from the bottom of the column 101′ to the top of the column 101″, or in the counter-current direction, followed by flowing liquid from the top of the column 101′ to the bottom 101″ by using the tubing 108. Additionally, the re-circulation flow path 220 may be realized by the tubing 108 as well. Such a system is schematically illustrated in FIG. 3.

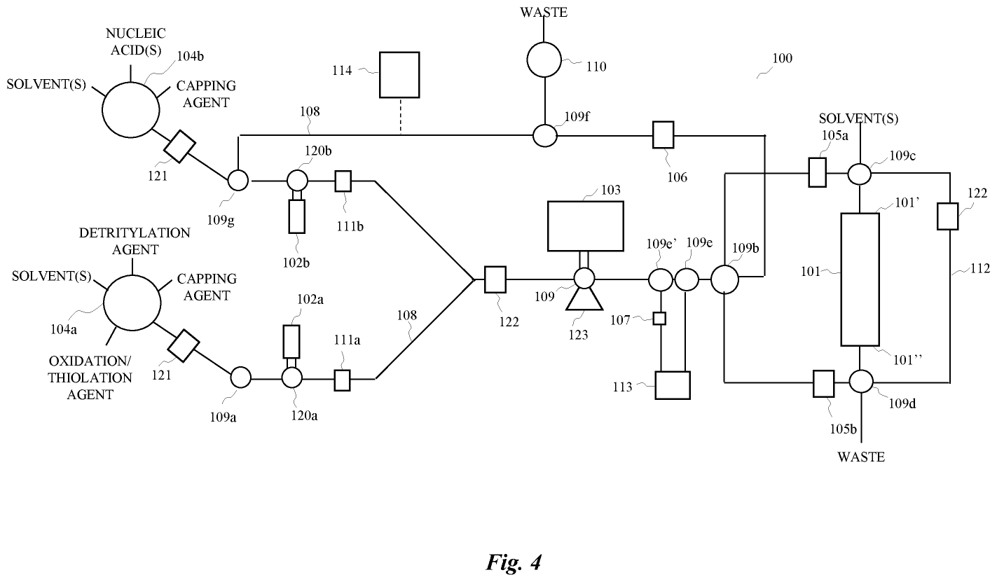

The system 100 may further comprise a column bypass 112 arranged in fluid communication with the tubing 108 and the column 101. A column bypass 112 is schematically illustrated in the system 100 in FIG. 4. A solvent inlet, and a waste outlet may be arranged in fluid communication with the column 101 and with the fluid bypass 112. The solvent inlet may be arranged upstream the column 101, and the waste outlet may be arranged downstream the column 101 or vice versa. The column bypass 112 may be arranged to recirculate liquid(s) in the system 100 without passing the column 101, for example during activation, washing, etc.

A system 100 according to one illustrated in any of FIGS. 2-4 may further comprise pressure sensor(s) 111a; 111b arranged downstream and in fluid communication with the reservoir(s) 104a; 104b. The system 100 may further comprise flow re-directing valves 120a; 120b in fluid communication with the first 102a, and the second pump 102b. The flow re-directing valves 120a; 120b may be arranged to direct the flow in and out of the respective pumps 102a; 102b. The flow re-directing valves 120a; 120b may be rotary valves, or solenoid valves or another suitable type of valves.

The pressure may vary during a synthesis process due to, for example, different flow rates, expansion of the resin, use of certain solvents, size of the equipment components such as tubing, etc. The liquid flowrate is arranged to be adjusted and regulated by pumps 102a; 102b in communicative connection with the processor 114 during use of the system 100. The regulation can be based on the pressure (in addition to other regulating parameters) so that the pressure inside the system 100 is kept within predetermined limits. Such predetermined limits depend on the process and the components of the system 100 and is to be determined by a person skilled in the art.

The system 100 may further comprise filters in the form of for example inlet filter(s) 121 and in-line filter(s) 122. The inlet 121 and in-line 122 filters may be arranged as schematically illustrated in FIG. 4, but other arrangements are also possible. An inlet filter 121 may be arranged at the tip(s) of inlet(s) to the system 100 in order to prevent undesired particles etc. from entering the system 100. In-line filter(s) 122 may be arranged to filter off unwanted components such as e.g. precipitates that have formed during a process. An in-line filter 122 can for example be placed upstream or downstream the pump(s) 102a;102b, or at the column bypass 112, or at any other suitable position in the system 100.

The valves 109a-f may be any type of valves that the skilled person finds suitable for a flow-through oligonucleotide synthesis system, it may for example be rotary valves, or solenoid valves or another suitable type of valves.

The valves 109a-f in the system 100 and/or flow re-directing valves 120a-b are arranged to direct the flow in the system 100, and they are in fluid communication with the tubing 108 and the reservoir(s) 104a; 104b. The valves 109a-f and/or flow re-directing valves 120a-b are controlled by and in communicative communication with the processor 114. The valves 109a-f in the system 100 and/or flow re-directing valves 120a-b are each arranged to be automated and controlled by the processor 114. The valves 109a-f and/or flow re-directing valves 120a-b may be regulated depending on the feedback to the system 100 from the monitoring. Such regulation is provided by the processor 114 in e.g. a computer that is in communicative connection with the system 100.

The detector 106 may be any type of detector, such as a UV/Vis detector, a fluorescence detector, a NIR detector, etc. In one embodiment the detector 106 is a spectroscopy detector, preferably a NIR detector. The detector 106 is arranged downstream the column 101. The detector 106 is in communicative communication with the processor 114. The system 100 may comprise more than one detector 106.

The system 100 may comprise more than one column 101, e.g. at least two columns 101 that can be connected in parallel. The use of more than one column 101 facilitates automated sequential synthesis of other sequences, synthesis in different scales, optimization of parameters for process development, etc.

While two reservoir(s) 104a;104b are illustrated in FIGS. 2-4 it should be understood that multiple reservoirs, e.g. each containing different types of solvents, sequence units, monomers, nucleic acid(s), different agents, reagents etc., could be used instead. It should further be understood that the different components of the system 100 such as the detector 106, the gas bubble detector 107, the pumps 102a; 102b, the temperature device 103, etc., could be arranged at different positions in the system 100. It should be noted that the FIGS. 2-4 merely show a few examples of possible configurations of the components in the system 100.

In one embodiment of the invention the system 100 further comprises
a temperature regulating device 103 arranged upstream the column 101 and downstream the at least first reservoir 104a;
a first temperature sensor 105a; and
a second temperature sensor 105b.

The first temperature sensor 105a is arranged upstream the column 101 and downstream the temperature regulating device 103, and the second 105b temperature sensor arranged downstream the column 101, as illustrated in FIGS. 2-4.

The temperature regulating device 103 can be arranged to either heat or cool, or both, a liquid, a liquid mixture or a synthesis liquid mixture etc. in the system 100. The liquid may comprise solvent(s), and/or reagents, and/or nucleic acid(s), and/or capping agent(s), etc. The temperature regulating device 103 is arranged upstream the column 101 and downstream the reservoirs 104a; 104b so that the liquid may be heated prior to reaching the column 101.

The system 100 may further comprise one or more flow paths (not illustrated) realized by tubings that are arranged to flow liquid having different temperatures through the system 100 and over the column 101. Such flow paths are in fluid communication with at least the temperature regulating device 103, the column 101, and the reservoir(s) 104a-b.

In one embodiment of the invention the system 100 further comprises a temperature regulating device bypass 123, as schematically illustrated in FIG. 4. The temperature regulating device bypass 123 is arranged in fluid communication with the temperature regulating device 103. The temperature regulating device bypass 123 may be arranged to bypass the synthesis liquid from the temperature regulating device 103, so that it does not pass the temperature regulating device 103.

In one embodiment of the invention the system 100 further comprises a separately defined mixing chamber 113 arranged in fluid communication with the at least first reservoir 104a, and a gas bubble detector 107 arranged upstream the separately defined mixing chamber 113, as illustrated in FIGS. 2-4.

The separately defined mixing chamber 113 may be used to activate nucleoside(s) or nucleotide(s) in the system 100. It is arranged in fluid communication with the at least first reservoir 104a so that the system 100 may be arranged to flow nucleoside(s) or nucleotide(s) and at least one activating agent(s) from the at least first reservoir 104a to the separately defined mixing chamber 113 by opening at least the first 109a and fifth 109e valve.

A gas bubble detector 107 may be arranged upstream the separately defined mixing chamber 113. The gas bubble detector 107 is in communicative communication with the processor 114. The gas bubble detector 107 can be arranged to detect the presence of a gas bubble as an indication that the mixing chamber is close to empty or emptied. The gas bubble detector 107 may be arranged upstream the separately defined mixing chamber 113 and downstream a second fifth valve 109e′ that is in fluid communication with the rest of the system 100.

In one embodiment of the invention the detector 106 is selected from e.g., a UV/Vis light detector, a fluorescence detector, and an NIR detector. The system 100 may be arranged to during use of the system 100 continuously monitor the progress of an oligonucleotide synthesis process or to monitor specific reaction steps, at specific time points, etc.

In a third aspect there is a method for performing flow-through solid-phase oligonucleotide synthesis 300 using a system 100 for flow-through solid-phase oligonucleotide synthesis. The system 100 comprises at least one column 101 arranged to be packed with synthetic resin; at least a first pump 102a, at least a first reservoir for monomers and reagents 104a; at least one detector 106, and at least one processor 114, wherein tubing 108 connects the first reservoir 104a to the column 101. The first pump 102a and valves 109a-d are arranged to direct the flow in the system 100. The processor 114 is in communicative connection at least with the first pump 102a, the valves 109a-d, and the detector 114. A synthetic resin is arranged in the column 101, and a first sequence unit is attached to the synthetic resin via chemical linkage. The method 300 comprises at least one step of reconsolidation 303, during which the system 100 is arranged to first fluidize the synthetic resin arranged in the column 101 by flowing a solvent from the bottom of the column 101′ to the top 101″ via the fluidizing flow path 200 in a fluidizing step 303′. The fluidizing step 303′ is followed by a repacking step 303″ in which the system 100 is arranged to repack the synthetic resin arranged in the column 101 by flowing a solvent from the top of the column 101″ to the bottom 101′ using the repacking flow path 210.

In one embodiment of the invention there is a method 300 for performing flow-through solid-phase oligonucleotide synthesis using a system 100 for flow-through oligonucleotide synthesis. The method 300 comprises the steps of:
Detritylation step 301: wherein the system 100 is arranged to flow an acidic solution comprising a detritylation agent through the tubing 108 from the first reservoir 104a to the column 101 by opening at least the first 109a, the second 109b, and the third 109c valve;
First washing step 302: wherein the system 100 is arranged to flow solvent through the tubing 108 from the first reservoir 104a to the column 101 by opening at least the first 109a, the second valve 109b, and the third 109c valve;
First reconsolidation step 303: wherein the system 100 is arranged to first fluidize the synthetic resin arranged in the column 101 by flowing a solvent from the bottom of the column 101′ to the top 101″ via the fluidizing flow path 200 in a fluidizing step 303′. The fluidizing step 303′ is followed by a repacking step 303″ wherein the system 100 is arranged to repack the synthetic resin arranged in the column 101 by flowing a solvent from the top of the column 101″ to the bottom 101′ using the repacking flow path 210;
Reaction step 320: wherein the system 100 is arranged to flow at least one sequence unit through the tubing 108 from the first reservoir 104a to the column 101 by opening at least the first 109a, the second 109b, and the third 109c valve. The second part of the reaction step 320 is a second washing step 305 wherein the system 100 is arranged to flow solvent through the tubing 108 from the first 104a reservoir to the column 101 by opening at least the first 109a, the second 109b, and the third 109c valve;
Oxidation/thiolation step 330: wherein the system 100 is arranged to flow an oxidation or thiolation agent through the tubing 108 from the first reservoir 104a to the column 101 by opening at least the first 109a, the second 109b, and the third 109c valve. The first part of the oxidation/thiolation step is followed by a third washing step 307 wherein the system 100 is arranged to flow solvent through the tubing 108 from the first 104a reservoir to the column 101 by opening at least the first 109a, the second 109b, and the third 109c valve; and
Full capping step 309: wherein the system 100 is arranged to flow a capping agent through the tubing 108 from the first reservoir 104a to the column 101 by opening at least the first 109a, the second 109b, and the third 109c valve. The first part of the capping step 309 is followed by a fourth washing step 307 wherein the system 100 is arranged to flow solvent through the tubing 108 from the first 104a reservoir to the column 101 by opening at least the first 109a, the second 109b, and the third 109c valve.

As described above, the system 100 may have more than one reservoir 104a, such as two reservoirs 104a; 104b, as illustrated in FIGS. 2-4. In such case the first reservoir 104a may be exchanged for the second reservoir 104b (or vice versa) in all steps of the method 300 described herein.

In one embodiment a second reconsolidation step is performed after the capping 340 (or after the fourth washing step 309). Additional reconsolidation steps may be performed at any time in a method 300 according to the invention, for example after any of the washing steps 302;305;307;309.

In one embodiment of the invention the flow rate during the fluidizing step 303′ is 25-75 cm/h, and the fluidizing step 303′ is continued for 0.25-0.75, or 0.25-1, or more column volumes. In one embodiment of the invention the flow rate during the repacking step 303″ is 100-200 cm/h, and the repacking step 303″ is continued for 0.25-0.75, or 0.25-1, or more column volumes.

The flow rate and the time period of the reconsolidation step 303 depend on the type of reaction, and/or the desired outcome, and/or the size (length and diameter) or volume and type of column 101, etc. In a method 300 according to the invention the system 100 is arranged to run the reconsolidation step 303 for a pre-determined time period using a pre-determined flow rate provided by the processor 114 determined based on the parameters described above.

In one embodiment of the invention the progress of the method 300 is monitored by at least one detector 106 arranged downstream the column 101.

In one embodiment of the invention the method 300 comprises an activation step of prior to the reaction step 320. In the activation step the system 100 is arranged to activate the at least one nucleotide or nucleoside by providing an activating agent to the at least one sequence unit. The activation can be performed in the tubing 108 of the system 100, or in a separately defined mixing chamber 113. The separately defined mixing chamber 113 is arranged in fluid communication with the first reservoir 104a. In one embodiment the system 100 is arranged to monitor the leaving of the activated at least one sequence unit from the separately defined mixing chamber 113 using a gas bubble detector 107 arranged upstream the separately defined mixing chamber 113.

In one embodiment of the invention the method 300 comprises an additional step prior to the reaction step 320 wherein the solution comprising at least one second sequence unit, or nucleotide or nucleoside, is heated prior to enter the column 101. In the heating step the system 100 is arranged to flow the solution comprising the at least one second sequence unit, or nucleotide or nucleoside, through a temperature regulating device 103. The temperature regulating device 103 is arranged upstream the column 101 and downstream the first reservoir 104a.

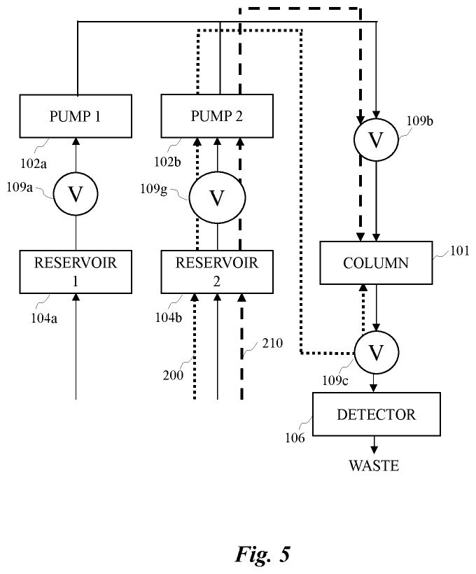

A typical flow-through oligonucleotide synthesis using a system 100 according to the invention is illustrated in the block scheme in FIG. 5 and in the flow-chart in FIG. 1a. As discussed above, the synthesis can be divided in the following steps:
Detritylation 301 The synthesis starts by arranging the system 100 to provide at least one detritylation agent from the first reservoir 104a to the column 101 by using at least the first valve 109a, and the first pump 102a. After the detritylation step 301 is finished, as determined for example by a conditional threshold detected by the detector 106, a solvent is passed through the system 100 in order to wash away any residues from the detritylation step 301. The system 100 is arranged to provide solvent from the first reservoir 104a (or second reservoir 104b) by using at least the first valve 109a, and the first pump 102a. After the first washing step 302 is finished, as determined for example by a conditional threshold detected by the detector 106 a reconsolidation step 303 follows.
Reconsolidation 303 In the reconsolidation step 303 solvent is first passed through the column 101 in a counter current direction, i.e. from the bottom of the column 101″ to the top 101′. Solvent is passed in counter-current direction, i.e. from the top of the column 101′ to the bottom 101″, through the column 101 until the synthetic resin, or resin bed, is at least partly fluidized, such as 90%, or 95%, or 99% fluidized, or until at least 1% of the resin is no longer in contact with the bottom of the column 101″. The synthetic resin is fluidized when the resin is not in contact with the bottom of the column 101″, hence the resin is fluidized when there is space not filled with resin (e.g. a gap, >1%) in between the synthetic resin and the bottom of the column 101″. Thus, until there is a fluidized bed arranged in the column 101. In other words, in the reconsolidation step 303 the system 100 is arranged to provide solvent from the first reservoir 104a to the column 101 using first the fluidizing flow path 200 followed by the repacking flow path 210. After the reconsolidation step 303 is finished the resin is evenly packed in the column 101. In the case that a transparent column 101 is used in the system 100 this can be visually observed.
Reaction step 320 The reaction step comprises a coupling step 304 followed by a second washing step 305. In the coupling step 304 the system 100 is arranged to pass at least one sequence unit, or nucleoside or nucleotide through the column 101 from the second reservoir 104b to the column 101 by using at least the seventh valve 109g, and the second pump 102b. After the coupling step 304 is finished, as determined for example by a conditional threshold detected by the detector 106 a second washing step 305 follows. During the second washing step 305 a solvent is passed through the system 100 in order to wash away any residues from the coupling 304. The system 100 is arranged to provide solvent from the second reservoir 104a (or first reservoir 104a) by using at least the seventh 109g (or first 109a) valve, and the second pump 102b (or first pump 102a). After the second washing step 305 is finished, as determined for example by a conditional threshold detected by the detector 106 an oxidation step 330 follows. The coupling step 304 may be repeated one or several times, so that the at least one sequence unit, or nucleotide or nucleoside may be re-circulated through the column 101.
Oxidation/thiolation step 330 An oxidation or thiolation step 330 follows the coupling step 320. The system 100 is arranged to provide an oxidating/thiolation agent from the first reservoir 104a to the column 101 by using at least the first valve 109a, and the first pump 102a. After the oxidation step 306 is finished, as determined for example by a conditional threshold detected by the detector 106, a solvent is passed through the system 100 in order to wash away any residues from the oxidation in a third washing step 307. The system 100 is arranged to provide solvent from the first reservoir 104a (or second reservoir 104b) by using at least the first 109a (or seventh 109g) valve, and first 102a (or second 102b) pump. After the third washing step 307 is finished, as determined for example by a conditional threshold detected by the detector 106 a capping step 340 follows.
Capping 340 In the capping step 340 the system 100 is arranged to provide a capping agent from the second reservoir 104b to the column 101 by using at least the seventh valve 109g and second pump 102b. After the capping step 308 is finished, as determined for example by a conditional threshold detected by the detector 106, a solvent is passed through the system 100 in a fourth washing step 309 in order to wash away any residues from the capping step 308. The system 100 is arranged to provide solvent from the first reservoir 104a (or second reservoir 104b) by using at least the first 109a (or seventh 109g) valve, and first 102a (or second 102b) pump.

In one embodiment of the invention the reaction step 320 is preceded by an activation step. During the activation step the system 100 is arranged to activate the at least one nucleoside or nucleotide by providing an activating agent. In one embodiment the system 100 is arranged to activate the at least one sequence unit, or nucleoside or nucleotide in the tubing 108 of the system 100 by simultaneously flowing the activating agent and the at least one sequence unit, or nucleoside or nucleotide through the tubing 108 of the system 100. In one embodiment the system 100 is arranged to activate the at least one sequence unit, or nucleoside or nucleotide in the separately defined mixing chamber 113, such activation may be monitored by a detector. Already activated sequence unit, or nucleoside(s) or nucleoside(s) may also be used in a method 300 according to the invention, in such case no activation step is necessary.

The present invention is not limited to the above-described embodiments. Various alternatives, modifications and equivalents may be used. Therefore, the above embodiments should not be taken as limiting the scope of the invention, which is defined by the appending claims. Additionally, all embodiments, aspects, and examples may be combined with each other unless explicitly stated otherwise.

### EXAMPLES

HPLC analysis method and conditions. Column: Phenomenex Aeris Peptide XB-C18, 2.6 μm, 100 Å, 150×2.1 mm. Mobile phase buffer A: 100 mM hexylammonium acetate, pH 7, buffer B: 50% acetonitrile in A. Gradient: 0% B for 4 min, 0-30% B in 2 min, 30-80% B in 30 min, 80-100% B in 2 min, 100% B for 2 min. Flowrate: 0.25 ml/min. Column temperature: 50° C. UV absorbance was recorded at 260 nm.

### Example 1: With Reconsolidation Prior to the Coupling Step

An oligonucleotide synthesis was performed using phosphoramidite standard synthesis conditions (S. L. Beaucage, M. H. Caruthers, Tetrahedron Lett, Vol 22, Issue 20, pp 1859-1862, 1981) of a Test-13 oligonucleotide sequence using a commercially available polystyrene/DVB crosslinked resin pre-derivatized with T. A reconsolidation (fluidizing and repacking) step was performed prior to the coupling step.

### Specific Conditions:

1.25 equivalents of “amidite”
2 min coupling time

### Results:

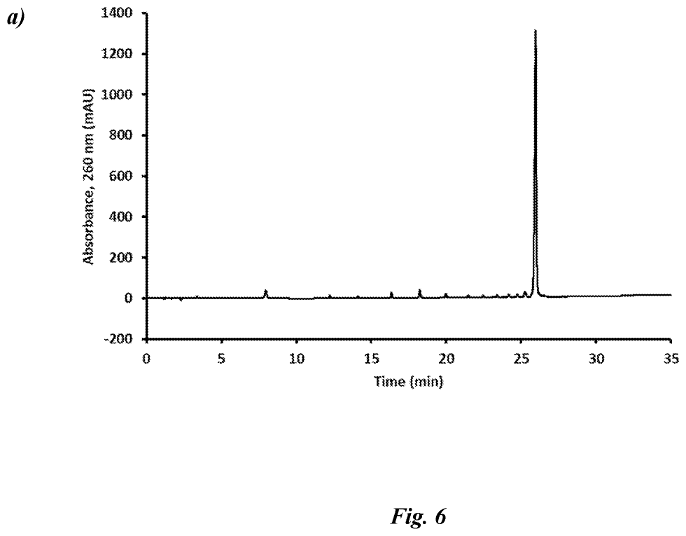

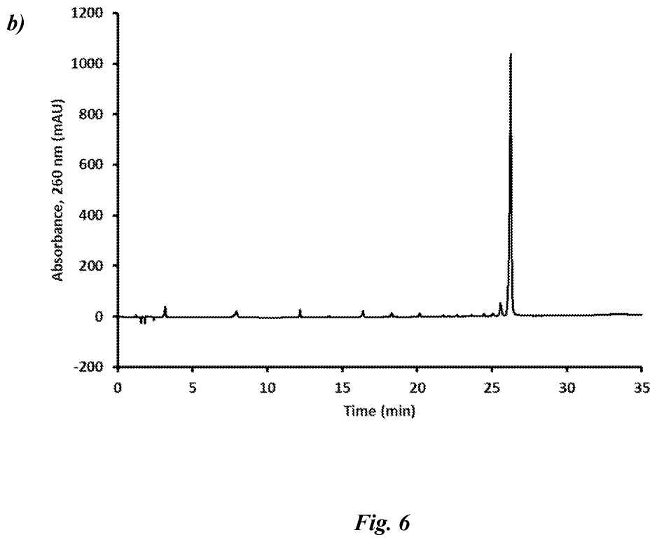

Total yield based on weight gain (after extensive drying), 84%.
Total yield (based on partial cleavage from resin and deprotection, A260 units), 77%.
Purity by HPLC, 84%.
An HPLC chromatogram of the obtained sequence can be seen in FIG. 6a.

### Example 2: Without Reconsolidation (Comparative Example)

An oligonucleotide synthesis was performed using phosphoramidite standard synthesis conditions (S. L. Beaucage, M. H. Caruthers, Tetrahedron Lett, Vol 22, Issue 20, pp 1859-1862, 1981) of a Test-13 oligonucleotide sequence using a commercially available polystyrene/DVB crosslinked resin pre-derivatized with T. A reconsolidation step was not performed prior to the coupling step.

### Specific Conditions:

1.25 equivalents of “amidite”
2 min coupling time

### Results:

Total yield based on weight gain (after extensive drying), 66%.
Purity by HPLC, 84%.
An HPLC chromatogram of the obtained sequence can be seen in FIG. 6b.

1.-30. (canceled)

31. A flow-through process for solid-phase oligonucleotide synthesis in which a first hydroxyl-protected sequence unit attached to a synthetic resin via chemical linkage is provided in a column, wherein the process comprises steps (i)-(iii), repeated at least once as a cycle, of
i. by detritylation, unprotecting the hydroxyl group of the first sequence unit attached to a synthetic resin;
ii. providing a liquid reaction solution comprising at least one second sequence unit; and
iii. passing the reaction solution over the synthetic resin with the at least one first sequence unit attached, whereby the at least one second sequence unit is coupled to the first sequence unit attached to the resin, and the reaction solution consolidates the resin to a more dense state in the column than provided in step (i);
within which process the step of removing a protecting group from the first sequence unit attached to a synthetic resin is performed in an acidic solution, and
wherein the process comprises at least one step of reconsolidating the resin.

32. The process according to claim 31, wherein the first and second sequence units are nucleotides or nucleoside based.

33. The process according to claim 31, in which a step of reconsolidating the synthetic resin is performed prior to the coupling step.

34. The process according to claim 31, wherein the step of reconsolidation comprises fluidizing the resin.

35. The process according to claim 31, in which the pKa of the acidic solution used in the detritylation step is 0.2-3.

36. The process according to claim 31, wherein the sequence units are activated in a separately defined mixing chamber.

37. The process according to claim 31, wherein the second sequence units are activated in the tubing of the system.

38. The process according to claim 31, wherein the reaction solution is passed over the resin at a flow rate and in a volume that allow for the at least one second sequence unit in the reaction solution to be in contact with at least 99% of the immobilized sequence unit(s).

39. The process according to claim 31, wherein heating is applied to the at least one second sequence unit comprised in the liquid reaction solution prior to being passed over the synthetic resin.

40. The process according to claim 39, wherein the at least one second sequence unit is heated at least 1° C. prior to being passed over the synthetic resin.

41. The process according to claim 31, wherein the progress of at least one step of the process is monitored by at least two detectors.

42. The process according to claim 31, wherein the progress of the process is continuously monitored.

43. The process according to claim 31, wherein the progress of the process is monitored by near-infrared (NIR) spectroscopy.

44. The process according to claim 31, wherein the process is linearly scalable.

45. The process according to claim 31, wherein the process uses software controlled real-time conditional monitoring that enables the use of Process Analytical Technology (PAT) to measure Critical Process Parameters (CPP) which affect Critical Quality Attributes (CQA).

46. The system for flow-through solid-phase oligonucleotide synthesis, wherein the system comprises at least one column arranged to be packed with synthetic resin; at least a first pump; at least a first reservoir for monomers and reagents; at least one detector, and at least one processor,
wherein tubing connects the first reservoir to the column, and wherein the first pump and valves are arranged to direct the flow in the system, and wherein the processor is in communicative connection at least with the first pump, the valves, and the detector,
wherein the system is arranged to provide a fluidizing flow path arranged to direct a flow of solvent from the first reservoir to the bottom of the column so that solvent pass through the column from the bottom of the column to the top, and
wherein the system is arranged to provide repacking flow path arranged to direct a flow of solvent from the first reservoir to the top of the column so that solvent pass through the column from the top to the bottom, and
wherein the system is arranged to in one synthesis cycle first flow solvent through the fluidizing flow path followed by flowing solvent through the repacking flow path.

47. The system according to claim 46, wherein when solvent passes through the bottom of the column to the top the third valve is arranged provide a waste outlet, and when solvent passes through the top of the column to the bottom the fourth valve is arranged to provide a waste outlet.

48. The system according to claim 46, wherein the fluidizing flow path and the repacking flow path are realized by the tubing.

49. The system according to claim 46, wherein the system further is arranged to provide a recirculation flow path, wherein the recirculation flow path is arranged to re-circulate solvents and reagents over the column.

50. The system according to claim 46, wherein the system further comprises a temperature regulating device (arranged upstream the column and downstream the at least first reservoir, a first temperature sensor arranged upstream the column and downstream the temperature regulating device, and a second temperature sensor arranged downstream the column.

51. The system according to claim 46, wherein the system further comprises a separately defined mixing chamber arranged in fluid communication with the at least first reservoir, and a gas bubble detector arranged upstream the separately defined mixing chamber.

52. A method for performing flow-through solid-phase oligonucleotide synthesis using a system for flow-through solid-phase oligonucleotide synthesis, wherein the system comprises at least one column arranged to be packed with synthetic resin; at least a first pump; at least a first reservoir for monomers and reagents; at least one detector, and at least one processor,
wherein tubing connects the first reservoir to the column, and wherein the first pump and valves are arranged to direct the flow in the system, and wherein the processor is in communicative connection at least with the first pump, the valves, and the detector, in which a synthetic resin is arranged in the column, and a first sequence unit is attached to the synthetic resin via chemical linkage,
wherein the method comprises at least one step of reconsolidation, during which the system is arranged to first fluidize the synthetic resin arranged in the column by flowing a solvent from the bottom of the column to the top via the fluidizing flow path in a fluidizing step, and
wherein the fluidizing step is followed by a repacking step in which the system is arranged to repack the synthetic resin arranged in the column by flowing a solvent from the top of the column to the bottom using the repacking flow path.

53. The method according to claim 52 wherein the method comprises the steps of:
Detritylation step: wherein the system is arranged to flow an acidic solution comprising a detritylation agent through the tubing from the first reservoir to the column by opening at least the first, the second valve and the third valve;
First washing step: wherein the system is arranged to flow solvent through the tubing from the first reservoir to the column by opening at least the first, the second valve and the third valve;
First reconsolidation step: wherein the system is arranged to first fluidize the synthetic resin arranged in the column by flowing a solvent from the bottom of the column to the top via the fluidizing flow path in a fluidizing step, wherein the fluidizing step is followed by a repacking step in which the system is arranged to repack the synthetic resin arranged in the column by flowing a solvent from the top of the column to the bottom using the repacking flow path;
Reaction step: wherein the system is arranged to flow at least one sequence unit through the tubing from the first reservoir to the column by opening at least the first, the second valve, and the third valve, after which there a second washing step wherein the system is arranged to flow solvent through the tubing from the first reservoir to the column by opening at least the first, the second valve, and the third valve;
Oxidation/thiolation step: wherein the system is arranged to flow an oxidation or thiolation agent through the tubing from the first reservoir to the column by opening at least the first, the second, and the third valve, after which there is a third washing step, wherein the system is arranged to flow solvent through the tubing from the first reservoir to the column by opening at least the first, the second valve, and the third valve; and
Capping step: wherein the system is arranged to flow a capping agent through the tubing from the first reservoir to the column by opening at least the first, the second, and the third valve, after which there is a fourth washing step, wherein the system is arranged to flow solvent through the tubing from the first reservoir to the column by opening at least the first, the second, and the third valve.

54. The method according to claim 52, wherein the progress of a synthesis performed according to the method is monitored by at least one detector arranged downstream the column.

55. The method according to claim 52, wherein the flow rate during the fluidizing step is 25-75 cm/h, and the fluidizing step is continued for 0.25-1 column volumes, and wherein the flow rate during the repacking step is 100-200 cm/h, and the repacking step is continued for 0.25-1 column volumes.

56. The method according to claim 52, wherein the method comprises a step of activation prior to the reaction step, wherein during the step of activation the system is arranged to activate the at least one sequence unit by providing an activating agent to the at least one sequence unit.

57. The method according to claim 56, wherein the system is arranged to activate the at least one sequence unit in the tubing of the system by simultaneously flowing the activating agent and the at least one sequence unit through the tubing of the system.

58. The method according to claim 56, wherein the at least one sequence unit is activated in a separately defined mixing chamber.

59. The method according to claim 58, wherein the activated at least one sequence unit leaving the separately defined mixing chamber is monitored by a gas bubble detector.

60. The method according to claim 52, wherein the method comprises an additional step prior to the reaction step, wherein the solution comprising at least one second sequence unit is heated prior to enter the column, wherein the heating step the system is arranged to pass the solution comprising the at least one second sequence unit through a temperature regulating device arranged upstream the column.
